(19) United States
(12) Patent Application Publication
(10) Pub. No.: US 2015/0094562 A1
(43) Pub. Date: Apr. 2, 2015

(54) MAGNETIC RESONANCE IMAGING WITH DYNAMIC INVERSION PREPARATION

(71) Applicant: General Electric Company, Schenecxtady, NY (US)

(72) Inventors: Christopher Judson Hardy, Niskayuna, NY (US); Glenn Scott Slavin, Silver Spring, MD (US); Jeffrey Alan Stainsby, Toronto (CA); Martin Andreas Janich, Garching b. Munchen (DE)

(73) Assignee: General Electric Company, Schenectady, NY (US)

(21) Appl. No.: 14/043,220

(22) Filed: Oct. 1, 2013

(51) Int. Cl. G01R 33/567; A61B 5/055; G01R 33/48

(52) U.S. Cl. CPC G01R 33/5673; G01R 33/4828; A61B 5/055
USPC 600/413; 324/322

(57) ABSTRACT

A magnetic field may be applied to a subject having a plurality of tissues, including first and second tissues, causing a net longitudinal magnetization in the tissues. An inversion radio frequency pulse may be generated to invert the longitudinal magnetization from the tissues. Heart-rate timing information associated with a current ECG of the subject may be measured, and an inversion time TI may be dynamically calculated based at least in part on the heart-rate timing information. An excitation radio frequency pulse may then be generated. The generation of the excitation radio frequency pulse may occur a period of time after the generation of the inversion radio frequency pulse, and the period of time may be based on the dynamically calculated inversion time TI. Magnetic resonance imaging data may then be acquired.

### BRIEF DESCRIPTION OF THE DRAWINGS

The present application is associated with US Publication No. 2008/0081986 entitled “Method and Apparatus for Generating a Magnetic Resonance Image” and published on Apr. 3, 2008. The entire contents of that publication are incorporated herein by reference.

### BACKGROUND

Magnetic resonance imaging (MRI) is a medical imaging modality that can create pictures of the inside of a human body without using x-rays or other ionizing radiation. MRI uses a powerful magnet to create a strong, uniform, static magnetic field (i.e., the “main magnetic field”). When a human body, or part of a human body, is placed in the main magnetic field, the nuclear spins that are associated with the hydrogen nuclei in tissue water and fat become polarized. This means that the magnetic moments that are associated with these spins become preferentially aligned along the direction of the main magnetic field, resulting in a small net tissue magnetization along that axis (the “z axis”, by convention). An MRI system also comprises components called gradient coils that produce smaller amplitude, spatially varying magnetic fields when current is applied to them. Typically, gradient coils are designed to produce a magnetic field component that is aligned along the z axis, and that varies linearly in amplitude with position along one of the x, y or z axes. The effect of a gradient coil is to create a small ramp on the magnetic field strength, and concomitantly on the resonant frequency of the nuclear spins, along a single axis. Three gradient coils with orthogonal axes are used to “spatially encode” the MR signal by creating a signature resonance frequency at each location in the body. Radio Frequency (RF) coils are used to create pulses of RF energy at or near the resonance frequency of the hydrogen nuclei. These coils are used to add energy to the nuclear spin system in a controlled fashion. As the nuclear spins then relax back to their rest energy state, they give up energy in the form of an RF signal. This signal is detected by the MRI system and is transformed into an image using a computer and known reconstruction algorithms.

MR images may be created by applying currents to the gradient and RF coils according to known algorithms called “pulse sequences”. A pulse sequence diagram may be used to show the amplitude, phase and timing of the various current pulses applied to the gradient and RF coils for a given pulse sequence. The selection of a pulse sequence determines the relative appearance of different tissue types in the resultant images, emphasizing or suppressing tissue types as desired. The inherent MR properties of tissue, most commonly the relaxation times T1 and T2, may be exploited to create images with a desirable contrast between different tissues. For example, in an MR image of a brain, gray matter may be caused to appear lighter or darker than white matter, according to the MRI system operator's choice of pulse sequence.

A pulse sequence may include a “spin preparation”, which is comprised of RF and gradient pulses that are played out (i.e., performed or applied) prior to the acquisition of MR data. A spin preparation may be used to control the appearance of a specific tissue type in an image, or to suppress signal from a certain tissue. Tissue suppression techniques are most commonly used for suppressing signal from fat. Multiple spin preparations are known that are able to suppress signal from fat, including CHESS (Chemical Shift Selective) pulses and Inversion Recovery preparations.

In certain clinical imaging applications, it is desirable to suppress the signal not only from fat tissue but also from another type of tissue in the same set of images. In cardiac MRI, for example, a paramagnetic contrast agent is used to visualize injured myocardial tissue. After a bolus of contrast agent is delivered intravenously, infarcted tissue retains a higher concentration of contrast agent for a longer period. This contrast agent shortens the T1 in the infarcted tissue, causing it to appear bright relative to healthy myocardium on T1-weighted images. Imaging the heart after a delay post injection of a contrast agent is called “myocardial delayed enhancement imaging”. Tissues that have a delayed hyper-enhancement are considered non-viable. In this type of imaging, it is desirable to choose a pulse sequence that can suppress the signal from healthy myocardium, so that the borders of the bright contrast-media-enhancing infarcted tissue may be clearly depicted. However, the presence of adjacent pericardial fat, which is also bright on a T1-weighted sequence, can negatively impact the identification of the infarct's borders.

In addition, MR image contrast changes can occur as a result of a subject's heart rate. Moreover, these contrast changes can make the MR image difficult to read. For example, a subject's heart rate might increase because he or she is nervous during an MRI procedure. Other subject's may suffer from arrhythmia which can result in unpredictable heart rate changes. Note that heart rate changes of +1-20% have been measured during a breath-hold scan in volunteers and can be even larger in cardiac patients. In some cases, an operator may manually attempt to adjust MR image timing to account for heart rate changes, but such an approach is prone to errors. Another approach is to use a myocardial delayed enhancement application (and IR prep pulse sequence) that triggers at every second heart beat. This may result in an improved IR prep contrast, but may also result in a decreased sensitivity to heart rate changes and longer total scan times.

It would therefore be desirable to provide systems and methods to facilitate an acquisition of MR images in an automated, accurate, and consistent manner.

### BRIEF DESCRIPTION OF THE DRAWINGS

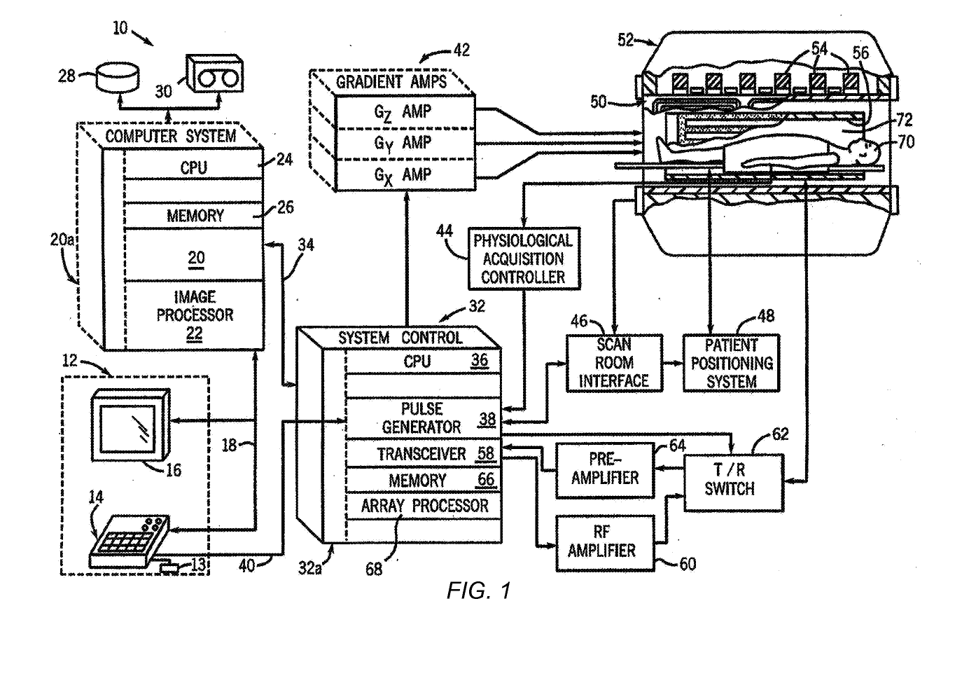

FIG. 1 is a schematic block diagram of a magnetic resonance imaging system according to some embodiments of the present invention.

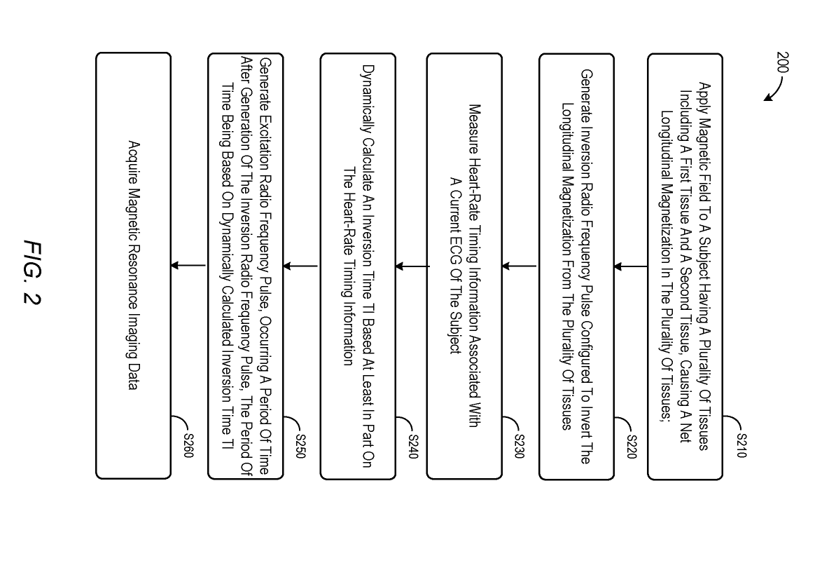

FIG. 2 illustrates a method that might be performed in accordance with some embodiments.

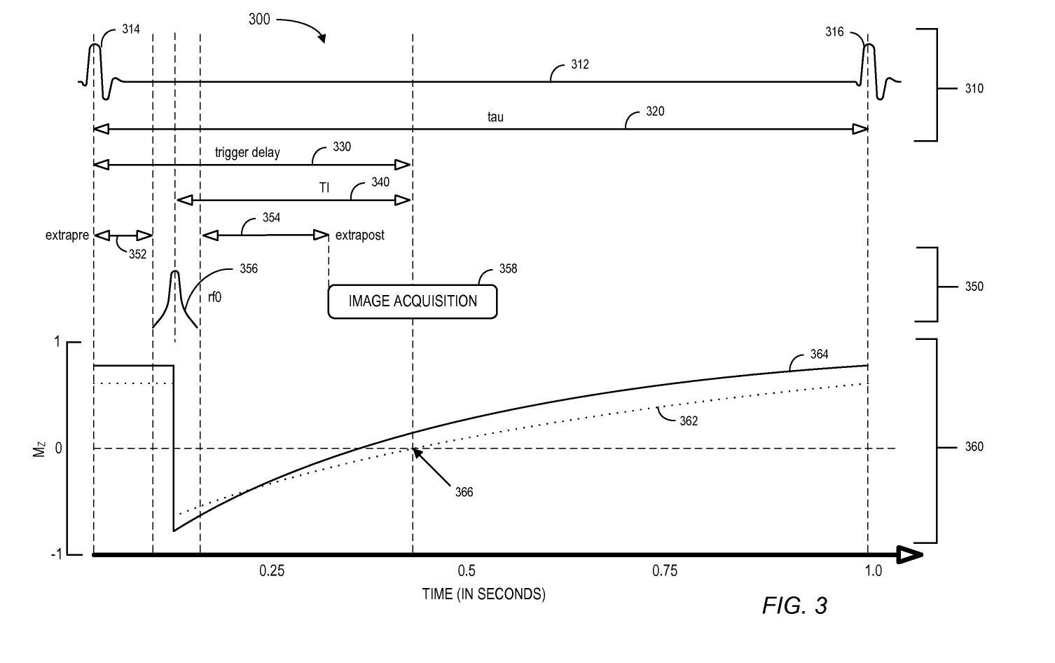

FIG. 3 is a sequence timeline associated with dynamic inversion preparation according to some embodiments.

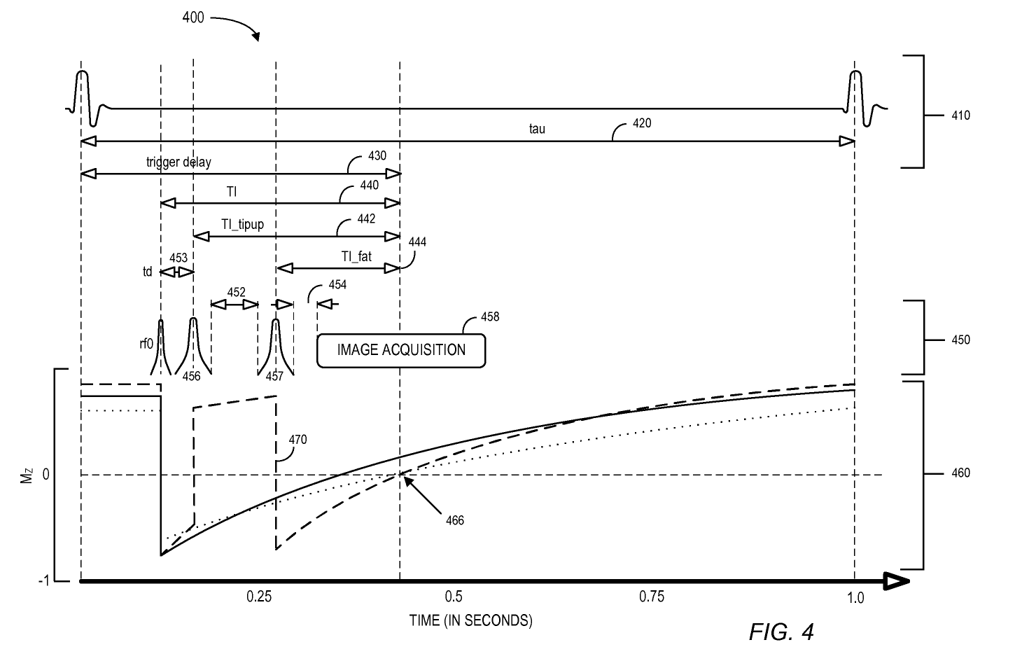

FIG. 4 is a sequence timeline associated with dynamic inversion preparation for fat tissue in accordance with some embodiments.

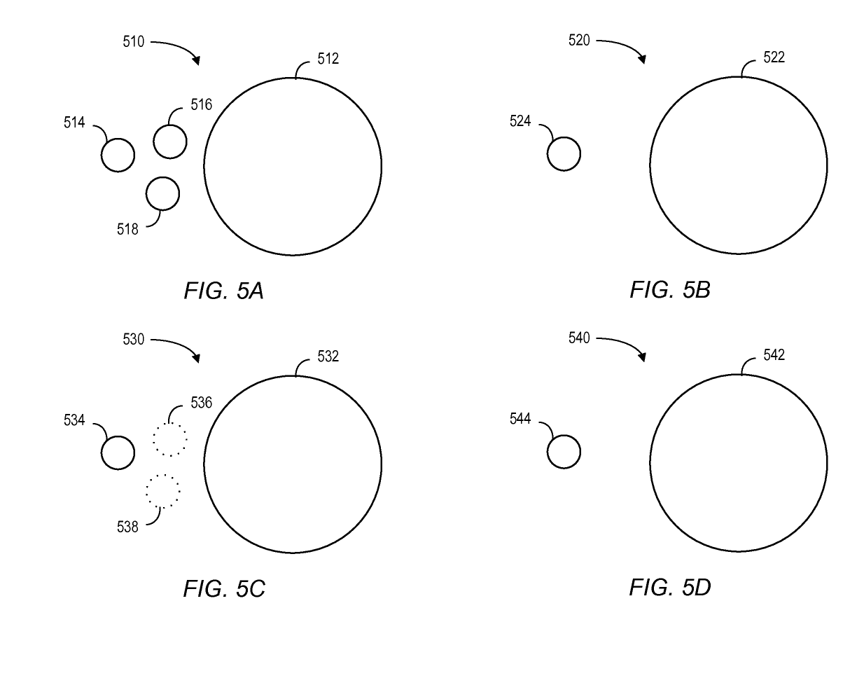

FIGS. 5A through 5D illustrate MR imaging situations associated with some embodiments of the present invention.

### DETAILED DESCRIPTION

In the following detailed description, numerous specific details are set forth in order to provide a thorough understanding of embodiments. However it will be understood by those of ordinary skill in the art that the embodiments may be practiced without these specific details. In other instances, well-known methods, procedures, components and circuits have not been described in detail so as not to obscure the embodiments.

To suppress signals from a particular type of tissue in a subject, a spin preparation may include a combination of a non-selective inversion RF pulse and two tissue-selective inversion RF pulses. According to some embodiments, the tissue-selective inversion RF pulses are inserted in the spin preparation between another non-selective inversion RF pulse (used to suppress another type of tissue in the subject) and an acquisition window of the pulse sequence. For example, a combination of fat-selective inversion RF pulses may suppress the fat signal without disturbing the desired T1 contrast that develops between the other (non-fatty) tissues of interest. The resultant spin preparation is comprised of: an inversion RF pulse configured to invert the longitudinal magnetization from all tissues including the fat tissue and a second tissue, followed by a first fat-selective inversion RF pulse, then a delay, followed by a second fat-selective inversion RF pulse such that fat is also nulled when the magnetization from the first tissue is nulled. In this application, “nulled” is used to mean that the longitudinal magnetization of a tissue is significantly reduced, such that it no longer detracts from a reader's ability to visualize the surrounding tissue. This does not require that data is acquired at exactly the null point of the tissue, but holds for a window of time around the null point. As will be described, according to some embodiments the timing of pulses within the pulse sequence are dynamically adjusted based on the subject's current ECG rate.

FIG. 1 is a schematic block diagram of an exemplary magnetic resonance imaging system 10. The operation of MRI system 10 is controlled from an operator console 12 that includes a keyboard or other input device 13, a control panel 14, and a display 16. The console 12 communicates through a link 18 with a computer system 20 and provides an interface for an operator to prescribe MRI scans, display the resultant images, perform image processing on the images, and archive data and images. The computer system 20 includes a number of modules that communicate with each other through electrical and/or data connections, for example such as are provided by using a backplane 20a. Data connections may be direct wired links, or may be fiber-optic connections or wireless communication links or the like. These modules include an image processor module 22, a CPU module 24 and a memory module 26. Memory module 26 may be, for example, a frame buffer for storing image data arrays as known in the art. In an alternative embodiment, the image processor module 22 may be replaced by image processing functionality on the CPU module 24. The computer system 20 is linked to archival media devices, such as disk storage 28 and tape drive 30 for storage of image data and programs, and communicates with a separate system control computer 32 through a high speed serial link 34. Archival media include but are not limited to: random access memory (RAM), read-only memory (ROM), electrically erasable programmable ROM (EEPROM), flash memory or other memory technology, compact disk ROM (CD-ROM), digital versatile disks (DVD) or other optical storage, magnetic cassettes, magnetic tape, magnetic disk storage or other magnetic storage devices, or any other medium which may be used to store the desired instructions and which can be accessed by computer system 20, including by internet or other computer network forms of access. The input device 13 can include a mouse, joystick, keyboard, track ball, touch activated screen, light wand, voice control, or any similar or equivalent input device, and may be used for interactive geometry prescription.

The system control computer 32 includes a set of modules in communication with each other via electrical and/or data connections 32a. Data connections 32a may be direct wired links, or may be fiber-optic connections or wireless communication links or the like. In alternative embodiments, the modules of computer system 20 and system control computer 32 may be implemented on the same computer systems or a plurality of computer systems. The modules of system control computer 32 include a CPU module 36 and a pulse generator module 38 that connects to the operator console 12 through a communications link 40. It is through link 40 that the system control computer 32 receives commands from the operator to indicate the scan sequence that is to be performed. The pulse generator module 38 operates the system components that play out (i.e., perform) the desired pulse sequence and produces data called RF waveforms which control the timing, strength and shape of the RF pulses to be used, and the timing and length of the data acquisition window. The pulse generator module 38 connects to a gradient amplifier system 42 and produces data called gradient waveforms which control the timing and shape of the gradient pulses that are to be used during the scan. The pulse generator module 38 may also receive patient data from a physiological acquisition controller 44 that receives signals from a number of different sensors connected to the patient, such as Electro Cardio Gram (ECG) signals from electrodes attached to the patient (e.g., to determine a subject's current ECG rate). The pulse generator module 38 connects to a scan room interface circuit 46 that receives signals from various sensors associated with the condition of the patient and the magnet system. It is also through the scan room interface circuit 46 that a patient positioning system 48 receives commands to move the patient table to the desired position for the scan.

The gradient waveforms produced by the pulse generator module 38 are applied to gradient amplifier system 42 which is comprised of Gx, Gy and Gz amplifiers. Each gradient amplifier excites a corresponding physical gradient coil in a gradient coil assembly generally designated 50 to produce the magnetic field gradient pulses used for spatially encoding acquired signals. The gradient coil assembly 50 forms part of a magnet assembly 52 that includes a polarizing magnet 54 and a whole-body RF coil 56. A patient or imaging subject 70 may be positioned within a cylindrical imaging volume 72 of the magnet assembly 52. A transceiver module 58 in the system control computer 32 produces pulses that are amplified by an RF amplifier 60 and coupled to the RF coils 56 by a transmit/receive switch 62. The resulting signals emitted by the excited nuclei in the patient may be sensed by the same RF coil 56 and coupled through the transmit/receive switch 62 to a preamplifier 64. The amplified MR signals are demodulated, filtered and digitized in the receiver section of the transceiver 58. The transmit/receive switch 62 is controlled by a signal from the pulse generator module 38 to electrically connect the RF amplifier 60 to the RF coil 56 during the transmit mode and to connect the preamplifier 64 to the coil during the receive mode. The transmit/receive switch 62 can also enable a separate RF coil (for example, a surface coil) to be used in either the transmit or receive mode.

The MR signals sensed by the RF coil 56 are digitized by the transceiver module 58 and transferred to a memory module 66 in the system control computer 32. Typically, frames of data corresponding to MR signals are stored temporarily in the memory module 66 until they are subsequently transformed to create images. Most commonly, a Fourier transform is used to create images from the MR data. These images are communicated through the high speed link 34 to the computer system 20 where it is stored in memory, such as disk storage 28. In response to commands received from the operator console 12, this image data may be archived in long term storage, such as on the tape drive 30, or it may be further processed by the image processor 22 and conveyed to the operator console 12 and presented on display 16.

Note that the recovery of longitudinal magnetization in an inversion preparation (IR prep) experiment may depend on T1 relaxation. Therefore, the amount of longitudinal magnetization which is recovered when the next IR prep pulse is applied may depend on the time that has passed before the previous IR prep pulse. In gated MR scans, the time between IR prep pulses may depend on a trigger signal (e.g., ECG). If the amount of signal recovery changes, then the timing of the signal may need to be changed in order to achieve the same IR prep contrast. According to some embodiments described herein, a measurement of the current heart rate is added to the scan and the current heart rate may be used to adapt the timing of IR prep pulses.

For example, FIG. 2 illustrates a method that might be performed by some or all of the elements of the system 10 described with respect to FIG. 1. The flow charts described herein do not imply a fixed order to the steps, and embodiments of the present invention may be practiced in any order that is practicable. Note that any of the methods described herein may be performed by hardware, software, or any combination of these approaches. For example, a computer-readable storage medium may store thereon instructions that when executed by a machine result in performance according to any of the embodiments described herein.

At S210, a magnetic field may be applied to a subject having a first tissue and a second tissue, and the magnetic field may cause a net longitudinal magnetization in the tissues. According to some embodiments, the first tissue may be normal myocardial tissue (which should preferably be nulled in the acquired MR image) and the second tissue may be myocardial infarct tissue (which should preferably be easily readable in the acquired MR image). At S220, an inversion RF pulse may be generated to invert the longitudinal magnetization from the first and second tissues.

At S230, heart-rate timing information associated with a current ECG of the subject may be measured. The heart-rate timing information may, for example, comprise “tau” representing a length of time between two heartbeats. At S240, an inversion time TI may be dynamically calculated based at least in part on the heart-rate timing information. According to some embodiments, this calculation may be described as:

TI=−T1*log [0.5+0.5*exp(−tau/T1)]

where T1 represents a tissue relaxation time and the log is the natural logarithm. At S250, an excitation RF pulse is generated a period of time after the generation of the inversion RF pulse, the period of time being based on the dynamically calculated inversion time TI.

At S260, magnetic resonance imaging data may be acquired. According to some embodiments, the generation of the excitation pulse and the acquisition of magnetic resonance imaging data are repeated to acquire multiple k-space lines of magnetic resonance imaging data. Moreover, the period of time may be such that k-space lines of magnetic resonance imaging data corresponding to a central aspect of k-space are acquired when a longitudinal magnetization of the first tissue is at or near a null point. Note that the generation of the excitation pulse and acquisition of magnetic resonance imaging data may be performed in accordance with: a fast gradient recalled acquisition, a balanced steady-state free precession acquisition, a spoiled gradient recalled acquisition, and/or any other type of readout.

According to some embodiments, the subject also includes fat tissue (which should preferably be nulled in the acquired MR image). In this case, two fat inversion RF pulses may be generated to invert the longitudinal magnetization from the fat tissue. Moreover, a fat inversion time TI_{fat} may be dynamically calculated based at least in part on the heart-rate timing information. In this case, the generation of the excitation RF pulse may occur a period of time after the generation of the second fat inversion RF pulse, and the period of time may be based on the dynamically calculated fat inversion time TI_{fat}. For example, TI_{fat} may be calculated as follows:

TI_{fat}=−T1_{fat}*log [0.5+exp(−(TI−td)/T1_{fat})−exp(−TI/T1_{fat})+0.5*exp(−tau/T1_{fat})]

where T1_{fat} represents a fat tissue relaxation time, the log is the natural logarithm, and td represents the time between the non-selective inversion pulse and the first fat inversion RF pulse (as will be illustrated as element 453 in FIG. 4).

FIG. 3 is a sequence timeline 300 associated with dynamic inversion preparation according to some embodiments. The timeline 300 includes ECG information 310, RF information 350, and M_{Z} information 360. In particular, the ECG information 310 includes an ECG signal 312 indicating a subject's first QRS 314 and second QRS 316. The time between the first QRS 314 and the second QRS 316 is referred to herein as “tau” 320. According to some embodiments, the current heart rate may be measured by periodically checking the ECG signal 312 (e.g., every 1 millisecond) to determine whether a trigger has appeared. If a trigger has appeared, then the time since the previous trigger is known (variable “tau” 320 in seconds) and this may define the subject's current heart rate (HR) in beats per minute: HR=60/tau.

An inversion RF pulse 356 (rf0) may be generated prior to an image acquisition 358 that occurs a trigger delay 330 after the subject's first QRS 314. Moreover, the image acquisition 358 may occur an inversion time TI 340 after the peak of this inversion RF pulse 356. The time between the subject's first QRS 314 and the beginning of the inversion RF pulse 356 is referred to as “extrapre” 352 and the time between the end of the inversion RF pulse 356 and the start of image acquisition 358 is referred to as “extrapost” 354. According to some embodiments, the inversion RF pulse 356 is timed relative to the center of ky-readout or image acquisition 358 (inversion time TI 340) according to the following formula:

TI=−T1*log [0.5+0.5*exp(−tau/T1)]

with T1 representing the T1 relaxation time and the log being the natural logarithm. As a result, the M_{Z} of a first tissue 362 (e.g., normal myocardial tissue) may be near a null point 366 during the center of the image acquisition 358 (and thus be suppressed in the resulting MR image) while the M_{Z} of a second tissue 364 (e.g., myocardial infarct tissue) may not be near the null point 366 during the center of the image acquisition 358 (and thus not be suppressed in the resulting M_{Z} image).

In addition to suppressing normal myocardial tissue in a resulting MR image, some embodiments described herein may be used to suppress fat tissue in the image. For example, FIG. 4 is a sequence timeline 400 associated with dynamic inversion preparation for fat tissue in accordance with some embodiments. As before, the timeline 400 includes ECG information 410, RF information 450, and M_{Z} information 460. The ECG information 410 includes an ECG signal indicating a subject's first QRS and second QRS and the time between the two heartbeats is referred to as “tau” 420.

In addition to the rf0 inversion RF pulse, two fat-inversion RF pulses may be generated prior to an image acquisition 458: an rf_tipup RF pulse 456 and an rf_cssat RF pulse 457. The center of the image acquisition 458 may occur a trigger delay 430 after the subject's first QRS. Moreover, the image acquisition 458 may occur an inversion time TI 440 after the peak of the rf0 inversion RF pulse. Moreover, center of the image acquisition 458 may occur a TI_tipup inversion time 442 after the peak of the rf_tipup RF pulse 546 and a TI_fat inversion time 444 after the peak of the rf_cssat RF pulse 457. The time between the end of the rf_tipup RF pulse 456 and the beginning of the rf_cssat RF pulse 457 is referred to as “extrapre” 452 and the time between the end of the rf_cssat RF pulse 457 and the start of image acquisition 458 is referred to as “extrapost” 454. According to some embodiments, the fat tipup and fat inversion RF pulses may be timed according to:

TI_fat=−T1_fat*log [0.5+exp(−(TI−td)/T1_fat)−exp(−TI/T1_fat)+0.5*exp(−tau/T1_fat)]

where “td” is the delay of the fat rf tipup RF pulse 546 relative to the rf0 inversion RF pulse. Note that both times TI and TI_fat may depend on the subject's current heart rate (HR=60/tau). As a result, the M_{Z} of fat tissue 470 may be near a null point 466 during the center of the image acquisition 458 (and thus be suppressed in the resulting MR image).

Thus, spin preparation may be used to suppress signal from fat and/or other types of tissue with the above-described MR system 10, or any similar or equivalent system for obtaining MR images. In the example of FIG. 4, the pulse sequence 400 includes a spin preparation comprising an initial rf0 pulse, a first fat-selective inversion RF pulse 456, and a second fat-selective inversion RF pulse 457 which might suppress the MR signals from both fat and normal myocardial tissue. The image acquisition 458 may comprise a single excitation RF pulse and an acquisition window, or may comprise multiple excitation RF pulses and acquisition windows, as for example, in a fast gradient recalled echo (fGRE) acquisition. Spin preparation may be compatible with base pulse sequences such as, for example, a two dimensional (2D) fGRE sequence, a regular 2D or three dimensional (3D) gradient recalled echo (GRE) sequence (in which a single alpha pulse is played out and a single k-space line is acquired following the spin preparation), a fast 3D GRE sequence, a regular spin echo sequence, or a fast spin echo sequence. FIG. 4 shows a spin preparation that is comprised of RF pulses including an initial inversion RF pulse rf0, a first fat-selective inversion RF pulse 456, and a second fat-selective inversion RF pulse 457, and also shows the timing of these RF pulses relative to the image acquisition 458. To create an MR image, the sequence of RF pulses shown in pulse sequence 400, together with appropriate gradient waveforms, may be played out (i.e., performed or applied) repeatedly until enough data is acquired to reconstruct an image. Multiple frames of data corresponding to individual lines in k-space may be collected during each image acquisition 458 by playing out multiple excitation pulses and acquisition windows in the image acquisition 458.

The inversion RF pulse rf0 may be a non-selective 180 degree inversion pulse that inverts the longitudinal magnetization for all tissues. The inversion RF pulse rf0 is followed by fat-selective inversion RF pulse 456 and fat-selective inversion RF pulse 457 to impact the longitudinal magnetization of fat 470 (M_{Zfat}) (e.g., driving the spin population of the fat tissue into a state which has an equal number of spins aligned with and against the positive z axis (+z), so that there is no net fat magnetization along the z axis). The rate of recovery of fat magnetization is known. The timing of the fat-selective inversion RF pulse 457 may be chosen such that fat achieves its null at approximately the same time as the healthy heart tissue (the center of image acquisition 458). Preferably, an acquisition scheme will be used that acquires the central lines of k-space when both the fat tissue and the first tissue are at or near their null points. Examples of acquisition schemes that are compatible with this spin preparation are a “centric encoding scheme”, in which the central lines of k-space are acquired early in the base sequence or a “sequential encoding scheme”, in which the central lines of k-space are acquired near the middle of the base sequence. The null points of fat and the first tissue may be timed to coincide with the acquisition of the central lines of k-space by appropriate modification of the TI, and the timing of the fat-selective inversion pulse 457 TI_fat. Note that the dynamic timing of either TI or TI_fat might be performed or, according to some embodiments, the dynamic timing of both TI and TI_fat may be performed together.

FIG. 4 also graphs 460 the resulting longitudinal magnetization M_{Zfat} 470 in accordance with the spin preparation RF information 450 in accordance with an embodiment. The graph 460 in FIG. 4 shows the time evolution of the longitudinal magnetization from fat 470 as well as other tissues in response to the sequence of RF pulses 450. The initial inversion RF pulse rf0 inverts the magnetization from all tissues, including fat and other tissue. The first fat-selective inversion RF pulse 456 moves the longitudinal magnetization from fat (MZfat 470), while having minimal effect on the longitudinal magnetization from the other tissues. While the spins from fat and the other tissues relax to their rest state, and the longitudinal magnetization of fat 470 and the other tissues re-grow along +z. The second fat-selective inversion RF pulse 457 inverts the fat magnetization. After the fat-selective inversion RF pulse 457, the magnetization from fat 470 re-grows along +z. The timing for the fat-selective inversion RF pulse 457 may be chosen such that the null point for fat occurs at time 466, i.e., at approximately the same time as the null point for healthy tissue. The image acquisition 458 may be determined such that the central lines of k-space are acquired at or near time 466 when the magnetization from fat is at its null point and the healthy tissue is at its null point.

Computer-executable instructions for performing a spin preparation according to the above-described method may be stored on a form of computer readable media. Computer readable media includes volatile and nonvolatile, removable and non-removable media implemented in any method or technology for storage of information such as computer readable instructions, data structures, program modules or other data. Computer readable media includes, but is not limited to, random access memory (RAM), read-only memory (ROM), electrically erasable programmable ROM (EEPROM), flash memory or other memory technology, compact disk ROM (CD-ROM), digital versatile disks (DVD) or other optical storage, magnetic cassettes, magnetic tape, magnetic disk storage or other magnetic storage devices, or any other medium which can be used to store the desired instructions and which may be accessed by MRI system 10 (shown in FIG. 1), including by internet or other computer network forms of access.

As mentioned above, the spin preparation described with respect to FIGS. 2 through 4 is compatible with various base pulse sequences. In addition, the spin preparation may be applied in various imaging applications such as, for example, cardiac imaging, abdominal imaging, musculoskeletal imaging, or imaging for any other part of the body. Accordingly, the dynamic spin preparation described herein may be applied to null the signal from fat and/or other tissue for any MRI imaging application.

FIGS. 5A through 5D illustrate imaging situations associated with some embodiments of the present invention. In particular, FIG. 5A illustrates an imaging situation 510 associated with a large object 512 and three small objects 514, 516, 518, including a doped water object 516 and a fat object 518. The doped water object 516 mixed with relaxation contrast agent (e.g., Gadolinium) may have a shorter T1 relaxation time as compared to object 514. FIG. 5B illustrates an imaging result 520 associated with inversion preparation for water and fat at a heart rate of 60 beats-per-minute. The inversion leads to signal nulling such that only the large object 522 and one small object 524 are visible in the image (and no doped water object or fat object are present).

FIG. 5C illustrates an imaging result 530 with inversion preparation for water and fat anticipating a heart rate of 60 beats-per-minute, but in this example the subject actually had a heart rate of 100 beats-per-minute. As a result, in addition to a large object 532 and one small object 534, trace images of a doped water object 536 and a fat object 538 are visible in the imaging result 530 (illustrated with dashed lines in FIG. 5C). FIG. 5D illustrates an imaging result 540 with inversion preparation for water and fat anticipating a heart rate of 60 beats-per-minute, but again this example the subject actually had a heart rate of 100 beats-per-minute. In this case, however, TI and TI_{fat} were dynamically and automatically adjusted based on the subject's actual heart rate during the scanning process. As a result of the dynamic inversion, the imaging result 540 includes a large object 542, one small object 544, and no images of a doped water object or fat object are visible in the imaging result 540 despite the heart rate change.

Thus, some embodiments described herein may help make scans less sensitive to variations in a subject's heart rate which are commonly seen during MRI examinations. This may lead to a more wide use of IR prep for MR imaging.

The present invention has been described in terms of several embodiments solely for the purpose of illustration. Persons skilled in the art will recognize from this description that the invention is not limited to the embodiments described, but may be practiced with modifications and alterations limited only by the spirit and scope of the appended claims.

1. A method for generating a magnetic resonance image, the method comprising:
applying a magnetic field to a subject, the subject having a plurality of tissues including a first tissue and a second tissue, wherein the magnetic field causes a net longitudinal magnetization in the plurality of tissues;
generating an inversion radio frequency pulse configured to invert the longitudinal magnetization from the plurality of tissues;
measuring heart-rate timing information associated with a current ECG of the subject;
dynamically calculating an inversion time TI based at least in part on the heart-rate timing information;
generating an excitation radio frequency pulse, said generation of the excitation radio frequency pulse occurring a period of time after said generation of the inversion radio frequency pulse, the period of time being based on the dynamically calculated inversion time TI; and
acquiring magnetic resonance imaging data.

2. The method of claim 1, wherein the first tissue is associated with normal myocardial tissue and the second tissue is associated with myocardial infarct tissue.

3. The method of claim 1, wherein the heart-rate timing information comprises tau, representing a length of time between two heartbeats.

4. The method of claim 3, wherein said calculating comprises:
TI=−T1*log [0.5+0.5*exp(−tau/T1)],
where T1 represents a tissue relaxation time and the log is the natural logarithm.

5. The method according to claim 1, wherein generating the excitation pulse and acquiring magnetic resonance imaging data are repeated to acquire multiple k-space lines of magnetic resonance imaging data.

6. The method according to claim 5, wherein the period of time is such that k-space lines of magnetic resonance imaging data corresponding to a central aspect of k-space are acquired when a longitudinal magnetization of the first tissue is at or near a null point.

7. The method according to claim 1, wherein generating the excitation pulse and acquiring magnetic resonance imaging data are performed in accordance with at least one of:
(i) a fast gradient recalled acquisition, (ii) a balanced steady-state free precession acquisition, (iii) a spoiled gradient recalled acquisition, and (iv) any other type of readout.

8. The method of claim 1, wherein the subject further includes fat tissue and the method further comprises:
generating a first fat inversion radio frequency pulse configured to invert the longitudinal magnetization from the fat tissue;
generating a second fat inversion radio frequency pulse configured to invert the longitudinal magnetization from the fat tissue;
dynamically calculating a fat inversion time TI_{fat} based at least in part on the heart-rate timing information, wherein said generation of the excitation radio frequency pulse occurs a period of time after said generation of the second fat inversion radio frequency pulse, the period of time being based on the dynamically calculated fat inversion time TI_{fat}.

9. The method of claim 8, wherein said calculating the fat inversion time TI_{fat} comprises:
TI_{fat}=−T1_{fat}*log [0.5+exp(−(TI−td)/T1_{fat})−exp(−TI/T1_{fat})+0.5*exp(−tau/T1_{fat})],
where T1_{fat} represents a fat tissue relaxation time, the log is the natural logarithm, and td represents a time between the non-selective inversion radio frequency pulse and the first fat inversion radio frequency pulse.

10. A non-transitory, computer-readable medium storing instructions that, when executed by a computer processor, cause the computer processor to perform a method for generating a magnetic resonance image, the method comprising:
applying a magnetic field to a subject, the subject having a plurality of tissues including a first tissue and a second tissue, wherein the magnetic field causes a net longitudinal magnetization in the plurality of tissues;
generating an inversion radio frequency pulse configured to invert the longitudinal magnetization from the plurality of tissues;
measuring heart-rate timing information associated with a current ECG of the subject;
dynamically calculating an inversion time TI based at least in part on the heart-rate timing information;
generating an excitation radio frequency pulse, said generation of the excitation radio frequency pulse occurring a period of time after said generation of the inversion radio frequency pulse, the period of time being based on the dynamically calculated inversion time TI; and
acquiring magnetic resonance imaging data.

11. The medium of claim 10, wherein the first tissue is associated with normal myocardial tissue and the second tissue is associated with myocardial infarct tissue.

12. The medium of claim 10, wherein the heart-rate timing information comprises tau, representing a length of time between two heartbeats, and said calculating comprises:
TI=−T1*log [0.5+0.5*exp(−tau/T1)],
where T1 represents a tissue relaxation time and the log is the natural logarithm.

13. The medium according to claim 11, wherein generating the excitation pulse and acquiring magnetic resonance imaging data are performed in accordance with at least one of:
(i) a fast gradient recalled acquisition, (ii) a balanced steady-state free precession acquisition, (iii) a spoiled gradient recalled acquisition, and (iv) any other type of readout.

14. The medium of claim 10, wherein the subject further includes fat tissue and the method further comprises:
generating a first fat inversion radio frequency pulse configured to invert the longitudinal magnetization from the fat tissue;
generating a second fat inversion radio frequency pulse configured to invert the longitudinal magnetization from the fat tissue;
dynamically calculating a fat inversion time TI_{fat} based at least in part on the heart-rate timing information, wherein said generation of the excitation radio frequency pulse occurs a period of time after said generation of the second fat inversion radio frequency pulse, the period of time being based on the dynamically calculated fax inversion time TI_{fat}.

15. The medium of claim 14, wherein said calculating the fat inversion time TI_{fat} comprises:
TI_{fat}=−T1_{fat}*log [0.5+exp(−(TI−td)/T1_{fat})−exp(−TI/T1_{fat})+0.5*exp(−tau/T1_{fat})],
where T1_{fat} represents a fat tissue relaxation time, the log is the natural logarithm, and td represents a time between the non-selective inversion radio frequency pulse and the first fat inversion radio frequency pulse.

16. An apparatus for generating a magnetic resonance image, the apparatus comprising:
a magnetic resonance imaging assembly comprising a magnet, a plurality of gradient coils, a radio frequency coil, a radio frequency transceiver system, and a pulse generator module; and
a computer system coupled to the magnetic resonance imaging assembly and programmed to perform a pulse sequence comprised of:
an inversion radio frequency pulse configured to invert a longitudinal magnetization from a plurality of tissues in a subject including a first tissue and a second tissue;
an excitation radio frequency pulse occurring a period of time after said inversion radio frequency pulse, the period of time being an inversion time TI which was dynamically calculated based at least in part on a heartbeat of the subject; and
an acquisition window to acquire magnetic resonance imaging data.

17. The apparatus of claim 16, wherein the first tissue is associated with normal myocardial tissue and the second tissue is associated with myocardial infarct tissue.

18. The apparatus of claim 16, wherein the heart-rate timing information comprises tau, representing a length of time between two heartbeats and said calculating comprises:
TI=−T1*log [0.5+0.5*exp(−tau/T1)],
where T1 represents a tissue relaxation time and the log is the natural logarithm.

19. The apparatus according to claim 1, wherein generating the excitation pulse and acquiring magnetic resonance imaging data are performed in accordance with at least one of:
(i) a fast gradient recalled acquisition, (ii) a balanced steady-state free precession acquisition, (iii) a spoiled gradient recalled acquisition, and (iv) any other type of readout.

20. The apparatus of claim 16, wherein the subject further includes fat tissue and the pulse sequence further comprises:
first and second fat inversion radio frequency pulses configured to invert the longitudinal magnetization from fat tissue, wherein the excitation radio frequency pulse occurs a period of time TI_{fat} after the second fat inversion radio frequency pulse, the period of time being based on the heart-rate timing information.

21. The apparatus of claim 20, wherein calculating the fat inversion time TI_{fat} comprises:
TI_{fat}=−T1_{fat}*log [0.5+exp(−(TI−td)/T1_{fat})−exp(−TI/T1_{fat})+0.5*exp(−tau/T1_{fat})],
where T1_{fat} represents a fat tissue relaxation time, the log is the natural logarithm, and td represents a time between the non-selective inversion radio frequency pulse and the first fat inversion radio frequency pulse.

22. The apparatus of claim 20, wherein the inversion radio frequency pulse comprises a non-selective 180 degree pulse, and the first and second fat inversion radio frequency pulses comprise fat-selective 180 pulses.
